(19) United States
(12) Patent Application Publication
(10) Pub. No.: US 2022/0253734 A1
(43) Pub. Date: Aug. 11, 2022

(54) MACHINE LEARNING METHODS TO OPTIMIZE CONCRETE APPLICATIONS AND FORMULATIONS

(71) Applicant: Parham Aghdasi, Richmond, CA (US)

(72) Inventor: Parham Aghdasi, Richmond, CA (US)

(21) Appl. No.: 17/578,886

(22) Filed: Jan. 19, 2022

Related U.S. Application Data
(63) Continuation-in-part of application No. 17/528,184, filed on Nov. 16, 2021.
(60) Provisional application No. 63/114,519, filed on Nov. 16, 2020.

(51) Int. Cl. G06N 7/00; G06N 20/20; G06K 9/62; G01N 33/38

(52) U.S. Cl. CPC G06N 7/005; G06N 20/20; G01N 33/383; G06K 9/6278; G06K 9/6262

(57) ABSTRACT

A method comprising: pulling a set of customer data and augment it with data sets designed to optimized machine learning operations; Selecting specified machine learning techniques from a specified machine learning database; taking into account a set of worksite context parameters; and optimizing and adjusting a concrete mix in real time to be able to deliver concrete products that meet requirements of project.

### CLAIM OF PRIORITY

This application claims priority to U.S. patent application Ser. No. 17/528,184, filed on Nov. 16, 2021 and titled MACHINE LEARNING METHODS TO OPTIMIZE CONCRETE APPLICATIONS AND FORMULATIONS. This application is hereby incorporated by reference in its entirety.

United States Patent Application No. claims priority to U.S. Provisional Application No. 63/114,519, filed on 2020 Nov. 16 and titled MACHINE LEARNING METHODS TO OPTIMIZE CONCRETE APPLICATIONS AND FORMULATIONS. This application is hereby incorporated by reference in its entirety.

### BACKGROUND

Concrete is the most ubiquitous construction material and the second most used substance in the world after water. The needs of the cement and concrete industry today and more importantly in the near future, given the fact that there are limited sources of raw materials used in concrete such as fly ash, amorphous silica, sand, etc. and that cement production is responsible for a large amount of carbon emissions, motivate further innovation and optimization of new cements and concretes. This can be done by using new methods and technologies to address the global challenges that face these industries in the most economical and sustainable ways possible. Accordingly, new methods of robotics and machine learning are desired to optimize, discover, and develop more advanced, cost-effective, durable, and sustainable cements and concrete composites.

### SUMMARY OF THE INVENTION

In one aspect, a method comprising: pulling a set of customer data and augment it with proprietary datasets designed to optimized machine learning operations; selecting specified machine learning techniques from a specified machine learning database; taking into account a set of worksite context parameters; and optimizing and adjusting a concrete mix in real time to be able to deliver concrete products that meet requirements of project.

In another aspect, a method of optimizing a concrete formulation includes the step of providing a historical data set of previously sampled concrete mixes. With the historical data set, the method includes the step of generating and validating a set of machine learning models; obtaining data from a concrete mix for input into a computer system that generates a machine learning model. The data is obtained by: obtaining three samples to be tested at each of a 1-day age of the concrete mix, a 2-day of the concrete mix, a 7-day of the concrete mix, a 14-day of the concrete mix, a 28-day of the concrete mix and a 56-day of the concrete mix; implementing a resistivity test and a compression test on each concrete sample. For each sample resistivity test and compression test there is a digital recording the resistivity test result and compression test result in a computer readable medium. The method includes the step of implementing a fresh properties test on each sample and digitally recording the results of the fresh properties test in the computer readable medium. The method includes the step of generating a test result dataset comprising each result of each digital recording of each resistivity test result, each compression test result, and each fresh properties test result. The method includes the step of, with the test result dataset, applying the set of machine learning models the test result dataset and generating a prediction output for at least one concrete mixing parameter of a new concrete mix.

### BRIEF DESCRIPTION OF THE DRAWINGS

The present application can be best understood by reference to the following description taken in conjunction with the accompanying figures, in which like parts may be referred to by like numerals.

FIG. 1 illustrates an example system useful to optimize concrete applications and formulations, according to some embodiments.

FIG. 2 illustrates an example process of optimize concrete applications and formulations, according to some embodiments.

FIG. 3 illustrates an example process for obtain data for input into an AI/ML for concrete applications and formulations optimization, according to some embodiments.

FIG. 4 illustrates an example concrete ML optimization process, according to some embodiments.

FIG. 5 illustrates an example process for feeding the data into an ML model, according to some embodiments.

FIG. 6 illustrates an example set of prediction models for implementing concrete method optimizations, according to some embodiments.

FIG. 7 depicts an exemplary computing system that can be configured to perform any one of the processes provided herein.

The Figures described above are a representative set, and are not an exhaustive with respect to embodying the invention.

### DESCRIPTION

Disclosed are a system, method, and article of manufacture for machine learning methods to optimize concrete formulations and applications. The following description is presented to enable a person of ordinary skill in the art to make and use the various embodiments. Descriptions of specific devices, techniques, and applications are provided only as examples. Various modifications to the examples described herein can be readily apparent to those of ordinary skill in the art, and the general principles defined herein may be applied to other examples and applications without departing from the spirit and scope of the various embodiments.

Reference throughout this specification to “one embodiment,” “an embodiment,” “one example,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art can recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, and they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

### Definitions

The following definitions are provided by way of example and not of limitation.

Adam (Adaptive Moment Estimation) is an update to the RMSProp optimizer (e.g. RMSProp (for Root Mean Square Propagation) is also a method in which the learning rate is adapted for each of the parameters). In this optimization algorithm, running averages of both the gradients and the second moments of the gradients are used.

Artificial neural network (e.g. a neural network as used herein) can be composed of artificial neurons or nodes. An artificial neural network can be used for solving artificial intelligence (AI) problems. The connections of the biological neuron are modeled in artificial neural networks as weights between nodes. A positive weight reflects an excitatory connection, while negative values mean inhibitory connections. All inputs are modified by a weight and summed. This activity is referred to as a linear combination. Finally, an activation function controls the amplitude of the output. For example, an acceptable range of output is usually between 0 and 1, or it could be −1 and 1. These artificial networks can be used for predictive modeling, adaptive control, and applications where they can be trained via a dataset.

Cement is a binder that sets, hardens, and adheres to other materials to bind them together. Cement can be used to aggregate sand and gravel together. Cement mixed with fine aggregate produces mortar for masonry and/or with sand and/or gravel can be used to produce concrete. Cements can be inorganic (e.g. lime and/or calcium silicate based, etc.). Cement can be non-hydraulic or hydraulic.

Central composite design (CCD) is an experimental design, useful in response surface methodology, for building a second order (e.g. quadratic) model for the response variable without needing to use a complete three-level factorial experiment. After the designed experiment is performed, linear regression can be used, sometimes iteratively, to obtain results. Coded variables can be used when constructing this design. The design can include three distinct sets of experimental runs: 1) a factorial (e.g. fractional) design in the factors studied, each having two levels; 2) a set of center points, experimental runs whose values of each factor are the medians of the values used in the factorial portion. This point is often replicated in order to improve the precision of the experiment; 3) a set of axial points, experimental runs identical to the center points except for one factor (e.g. can take on values both below and above the median of the two factorial levels, and typically both outside their range). The factors can be varied in this way.

Concrete is a composite material composed of fine and coarse aggregate bonded together with a fluid cement paste that hardens/cures as a function of time.

Concrete maturity/age can be an index value that represents the progression of concrete curing. It can be based on an equation that takes into account concrete temperature, time, and strength gain. Concrete maturity can be an accurate way to determine real-time strength values of curing concrete.

Concrete slump test measures the consistency of fresh concrete before it sets. It is performed to check the workability of freshly made concrete, and therefore the ease with which concrete flows. It can also be used as an indicator of an improperly mixed batch. The concrete slump test is used to ensure uniformity for different loads of concrete under field conditions.

Deep-learning algorithms can be based on a set of algorithms that attempt to model high-level abstractions in data by using multiple processing layers, with complex structures or otherwise, composed of multiple non-linear transformations.

Drop out method can be used to reduce overfitting. At each training stage, individual nodes are either “dropped out” of the net (e.g. ignored) with probability 1−p or kept with probability p, so that a reduced network is left; incoming and outgoing edges to a dropped-out node are also removed. Only the reduced network is trained on the data in that stage. The removed nodes are then reinserted into the network with their original weights.

Gradient boosting is a machine learning technique used in regression and classification tasks, among others. It gives a prediction model in the form of an ensemble of weak prediction models, which are typically decision trees. When a decision tree is the weak learner, the resulting algorithm is called gradient-boosted trees; it usually outperforms random forest. A gradient-boosted trees model is built in a stage-wise fashion as in other boosting methods, but it generalizes the other methods by allowing optimization of an arbitrary differentiable loss function.

K-nearest neighbors algorithm (KNN) is a non-parametric classification method. In KNN classification, the output is a class membership. An object is classified by a plurality vote of its neighbors, with the object being assigned to the class most common among its k nearest neighbors (k is a positive integer, typically small). If k=1, then the object is assigned to the class of that single nearest neighbor. In KNN regression, the output is the property value for the object. This value is the average of the values of k nearest neighbors. KNN is a type of classification where the function is approximated locally and computation is deferred until function evaluation. Since this algorithm relies on distance for classification, if the features represent different physical units or come in vastly different scales then normalizing the training data can improve its accuracy dramatically.

Machine learning is a type of artificial intelligence (AI) that provides computers with the ability to learn without being explicitly programmed. Machine learning focuses on the development of computer programs that can teach themselves to grow and change when exposed to new data. Examples of machine learning techniques that can be used herein include, inter alia: decision tree learning, association rule learning, artificial neural networks, inductive logic programming, support vector machines, clustering, Bayesian networks, reinforcement learning, representation learning, similarity, and metric learning, and/or sparse dictionary learning.

Monte Carlo methods are a broad class of computational algorithms that rely on repeated random sampling to obtain numerical results. The underlying concept is to use randomness to solve problems that might be deterministic in principle.

One-hot encoding can be used method to deal with categorical data. An ML model(s) can use input variables that are numeric. In one example, the categorical variables can be transformed in a pre-processing part. Categorical data can be either nominal or ordinal. Ordinal data can have a ranked order for its values and can therefore be converted to numerical data through ordinal encoding.

Quadratic discriminant analysis (QDA) can assume that the measurements from each class are normally distributed. In QDA there is no assumption that the covariance of each of the classes is identical. When the normality assumption is true, the best possible test for the hypothesis that a given measurement is from a given class is the likelihood ratio test.

Random forests are an ensemble learning method for classification, regression and other tasks that operates by constructing a multitude of decision trees at training time. For classification tasks, the output of the random forest is the class selected by most trees. For regression tasks, the mean or average prediction of the individual trees is returned. Random decision forests can be used for correcting for decision trees' habit of overfitting to their training set.

Response surface methodology (RSM) explores the relationships between several explanatory variables and one or more response variables. RSM can use a sequence of designed experiments to obtain an optimal response. RSM can use a factorial experiment or a fractional factorial design. This may be sufficient to determine which explanatory variables affect the response variable(s) of interest. Once it is suspected that only significant explanatory variables are left, then a more complicated design, such as a central composite design can be implemented to estimate a second-degree polynomial model, which is still only an approximation at best. However, the second-degree model can be used to optimize (e.g. maximize, minimize, or attain a specific target for) the response variable(s) of interest.

Synthetic Minority Oversampling Technique (SMOTE) is type of data augmentation for a minority class. SMOTE can select examples that are close in the feature space, drawing a line between the examples in the feature space and drawing a new sample at a point along that line.

### Exemplary Systems

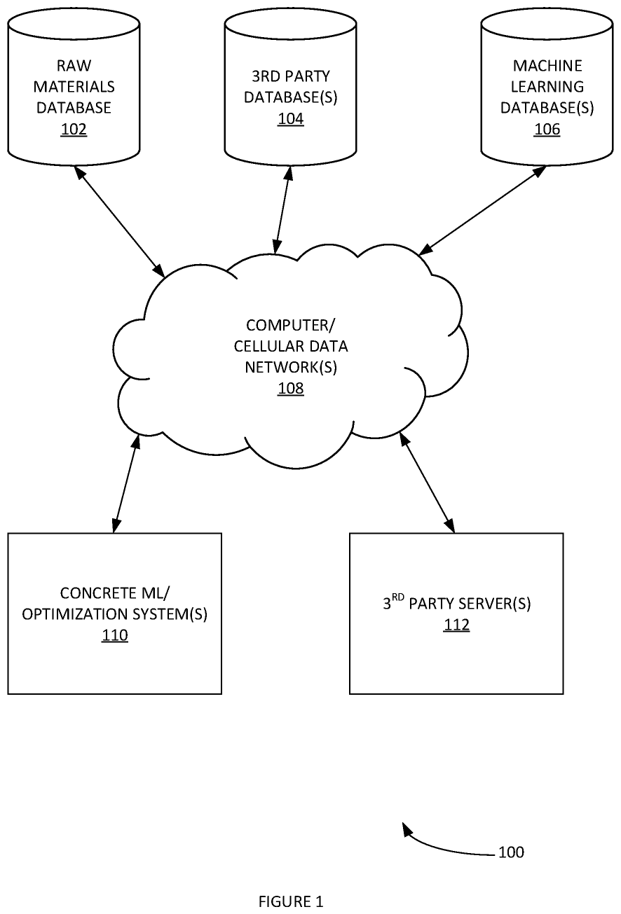

FIG. 1 illustrates an example system 100 useful to optimize concrete applications and formulations, according to some embodiments. System 100 can include computer/cellular data network(s) 108. Computer/cellular data network(s) 108 can communicatively couple the various systems within system 100. Computer/cellular data network(s) 108 can be various computer network(s) (e.g. the Internet, LANs, WANs, local Wi-Fi, cellular data networks, enterprise network, etc.).

Concrete ML/optimization system(s) 110 can include various machine-learning engines. Concrete ML/optimization system(s) 110 can utilize machine learning algorithms optimize concrete/cement formulae in a specified context of an application site. Machine learning is a type of artificial intelligence (AI) that provides computers with the ability to learn without being explicitly programmed. Machine learning focuses on the development of computer programs that can teach themselves to grow and change when exposed to new data. Examples of machine learning techniques that can be used herein include, inter alia: decision tree learning, association rule learning, artificial neural networks, inductive logic programming, support vector machines, clustering, Bayesian networks, reinforcement learning, representation learning, similarity, and metric learning, and/or sparse dictionary learning. Random forests (RF) (e.g. random decision forests) are an ensemble learning method for classification, regression, and other tasks, which operate by constructing a multitude of decision trees at training time and outputting the class that is the mode of the classes (e.g. classification) or mean prediction (e.g. regression) of the individual trees. RFs can correct for decision trees' habit of overfitting to their training set. Deep learning is a family of machine learning methods based on learning data representations. Learning can be supervised, semi-supervised or unsupervised.

Machine learning can be used to study and construct algorithms that can learn from and make predictions on data. These algorithms can work by making data-driven predictions or decisions, through building a mathematical model from input data. The data used to build the final model usually comes from multiple datasets. In particular, three data sets are commonly used in different stages of the creation of the model. The model is initially fit on a training dataset, which is a set of examples used to fit the parameters (e.g. weights of connections between neurons in artificial neural networks) of the model. The model (e.g. an artificial neural net or a naive Bayes classifier) is trained on the training dataset using a supervised learning method (e.g. gradient descent or stochastic gradient descent). In practice, the training dataset often consists of pairs of an input vector (or scalar) and the corresponding output vector (or scalar), which is commonly denoted as the target (or label). The current model is run with the training dataset and produces a result, which is then compared with the target, for each input vector in the training dataset. Based on the result of the comparison and the specific learning algorithm being used, the parameters of the model are adjusted. The model fitting can include both variable selection and parameter estimation. Successively, the fitted model is used to predict the responses for the observations in a second dataset called the validation dataset. The validation dataset provides an unbiased evaluation of a model fit on the training dataset while tuning the model's hyperparameters (e.g. the number of hidden units in an artificial neural network). Validation datasets can be used for regularization by early stopping: stop training when the error on the validation dataset increases, as this is a sign of overfitting to the training dataset. This procedure is complicated in practice by the fact that the validation dataset's error may fluctuate during training, producing multiple local minima. This complication has led to the creation of many ad-hoc rules for deciding when overfitting has truly begun. Finally, the test dataset is a dataset used to provide an unbiased evaluation of a final model fit on the training dataset. If the data in the test dataset has never been used in training (e.g. in cross-validation), the test dataset is also called a holdout dataset.

More specifically, concrete ML/optimization system(s) 110 can use proprietary datasets supplemented by partner data, as well as cutting edge AI tools. These can include, inter alia: such as model-based reinforcement learning, Bayesian optimization, model-based multi-armed bandits, convolutional neural networks, generative adversarial networks and other cutting-edge machine learning algorithms to predict the properties of millions of combinations of raw materials as well as organic and chemical admixtures and supplementary cementitious materials used in cement and concrete production, and producing and testing material structures and properties using robotic systems to quickly converge on optimal mixes or to discover new cements or concrete materials. This enables drastic improvements in cost and performance characteristics for each project.

In one example, concrete ML/optimization system(s) 110 provides service to two types of companies. Concrete ML/optimization system(s) 110 provides services to cement companies and concrete companies. It is noted that these can be a single or a plurality of entities.

In case of cement companies, concrete ML/optimization system(s) 110 uses customer data (e.g. 3rd party database(s) 104 obtained via 3rd party server(s) 112, etc.) augmented by example proprietary datasets (e.g. raw materials database 102, machine learning database(s) 106, etc.) as well as its ML/AI tools, to provide real-time feedback to cement production plants with the purpose of optimizing cement production, efficiency, and quality control in order to avoid undesirable cement properties before and after being used in the final concrete product.

It is noted that ML/optimization system(s) 110 can utilize a data-driven approach. ML/optimization system(s) 110 can manage proprietary data and a fully-automated robotic laboratory. ML/optimization system(s) 110 can collect large amounts of data (e.g. big data) for training, development, and testing its machine learning models. Finally, ML/optimization system(s) 110 can use its machine learning models backed by big data, generated through its fully-automated lab, to invent and discover new cements, concretes, and other construction materials to either license to other producers or to mass produce itself.

### Exemplary Methods

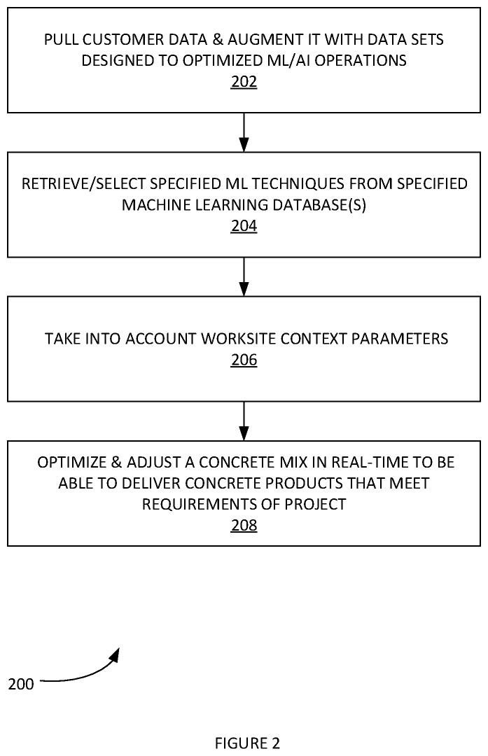

FIG. 2 illustrates an example process of optimize concrete applications and formulations, according to some embodiments. In step 202, process 200 pull customer data (e.g. of concrete companies) and augment it with data sets designed to optimized ML/AI operations. For example, process 200 can use ML/optimization system(s) 110 to pull customer data from 3rd party database(s) 104 and augment it with information from proprietary datasets (e.g. raw materials database 102, machine learning database(s) 106, etc.).

In step 204, process 200 can retrieve/select specified ML techniques from specified machine learning database(s) 106. Process 200 can select other ML/AI tools as well. These ML/AI tools can be dynamically selected to provide optimized real-time feedback to cement production plants. These ML/AI tools can be dynamically selected to provide optimized real-time feedback this time to concrete producers (e.g. concrete ready-mix companies or concrete precast plants) based on real-time observations from the time concrete is mixed until it is delivered to the site.

In step 206, process 200 can take into account worksite context parameters. For example, process 200 can obtain site-specific and other weather and environmental conditions.

In step 208, process 200 can optimize and adjust a concrete mix in real-time to be able to deliver concrete products that meet the requirements of the project and the goals and priorities of the customer as closely as possible. These could include but not limited to minimizing costs, minimizing cement consumptions, minimizing carbon footprint, maximizing, or achieving a certain over-night or early-age strength, maximizing durability, and the like using local raw materials available to producers. For example, if the goal is to reduce the amount of cement without compromising the final properties, ML/optimization system(s) 110 can enable concrete producers to use 10-20% less cement in their concrete, therefore, reducing costs as well as carbon footprint.

### Additional Methods and Embodiments

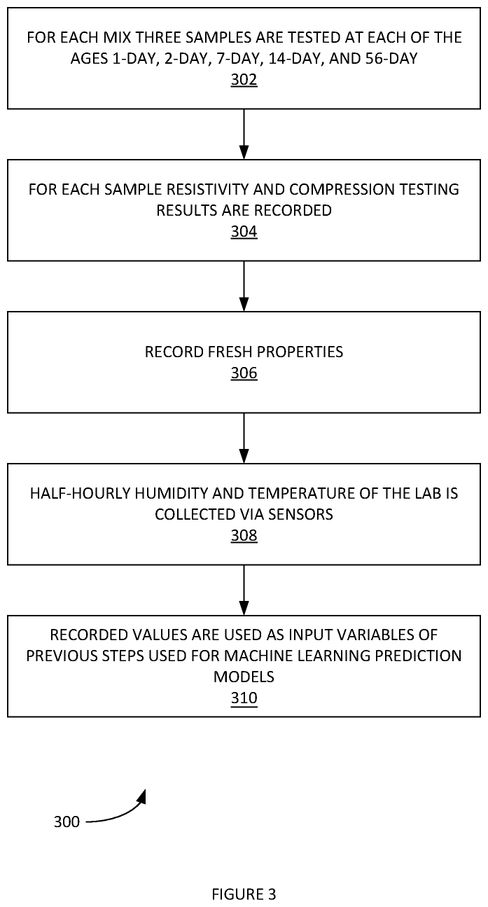

FIG. 3 illustrates an example process 300 for obtaining data for input into an AI/ML for concrete applications and formulations optimization, according to some embodiments. In step 302, for each mix three samples are tested at each of the ages 1-day, 2-day, 7-day, 14-day, 28-day, and 56-day. Resistivity and compression testing can be implemented on the concrete samples. In step 304, for each sample resistivity and compression testing results are recorded. In step 306, process 300 fresh properties are obtained and recorded. These can include, inter alia (e.g. recorded for each mix): slump, unit weight/density, air content, setting time, moisture content, and temperature and humidity during mixing. In addition, in step 308, process 300 can implement and obtain half-hourly humidity and temperature of the lab measures. It is noted that these measures can be obtained at other time intervals (e.g. every five minutes, etc.). These can be collected via sensors installed in the lab or on site. In step 310, the recorded values are used as input variables for machine learning prediction models. The data/information obtained from process 300 can be recorded in a computerized data store.

### Example Input and Output Variables

Example input variables are now discussed. The input variables can include, inter alia: weights of ingredients/raw materials; other raw material properties (e.g. aggregate gradations, cement properties, admixture properties, etc.);

absorption and moisture variables (e.g. sand free moisture (%), coarse aggregate free moisture (%), sand correction (lbs.), coarse aggregate correction (lbs.), sand absorption capacity (%), coarse aggregate absorption capacity, etc.);

temperature and humidity;

other weather conditions during mixing and curing by using weather stations and different sensors up until compressive strength testing with adjustable number of recordings during the day;

domain knowledge: this can include a list of variables drawn from the existing input variables which have proven effective in predicting the outputs in the literature is used to augment data inputs/features into the machine learning predictive models; etc.

Example output variables can include, inter alia: slump; density; air content; fresh concrete temperature; setting time; compressive strength at different ages; electrical resistivity at different ages; drying shrinkage values; cost; etc.

### Example Experimental Design

Various example designs of preliminary experiments are now discussed. It is noted that, as part of the data collection process, a Response Surface Design can be created. A Response Surface Design can be used for adjusting the balance of the data in the variable space and provides a measure of variance. The multi-variable Central Composite Design (CCD) uses 1) a full factorial design, 2) an axial design, and 3) center points. In one example, the portion coded value is set to alpha=(2k)0.25, where k is the number of design variables. For example, for k=6, alpha=2.828 noting the design code for the axial points. Then, the variables will have five different levels including −2.828, −1, 0, 1, 2.828. These values are provided by way of example and not of limitation.

### Example Prediction Models

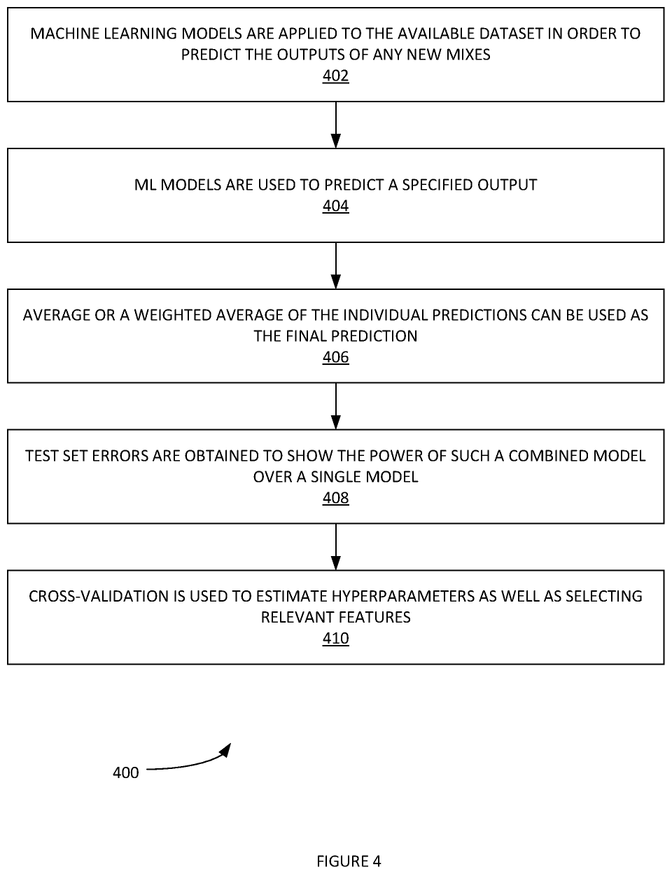

FIG. 4 illustrates an example concrete ML optimization process 400, according to some embodiments. In step 402, machine learning models are applied to the available dataset in order to predict the outputs of any new mixes. Because of the complexity of the data and difficulty of prediction, a combination of several regression models can be used. In step 404, these ML models are used to predict a specified output (e.g. slump, compressive strength at a certain age, etc.). Then, in step 406, an average or a weighted average of the individual predictions can be used as the final prediction. In step 408, test set errors are obtained to show the power of such a combined model over a single model. In step 410, cross-validation is used to estimate hyperparameters as well as selecting relevant features.

Prediction Models

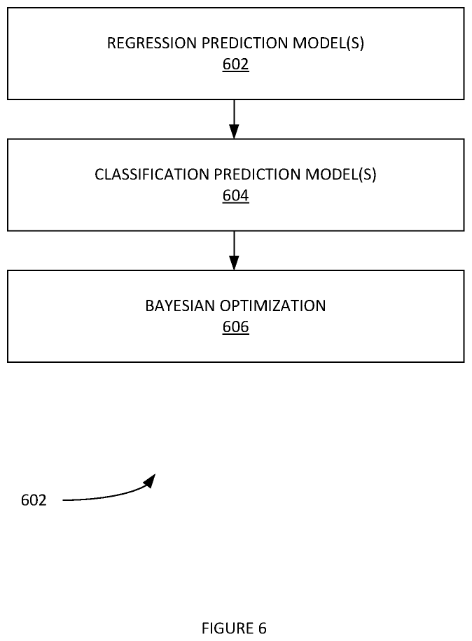

FIG. 6 illustrates an example set of prediction models for implementing concrete method optimizations, according to some embodiments. In step 602 process 600 can implement a regression model. Machine learning models are applied by process 600 to the available dataset(s) in order to predict the outputs of any new mixes. Process 600 can use a combination of several regression models. These regression models can be used to predict the output (e.g. slump, compressive strength at a certain age, etc.) Then, an average or a weighted average of the individual predictions can be used as the final prediction. Test set errors can be used to show the power of such a combined model over a single model. Cross-validation can be used to estimate hyperparameters as well as selecting relevant features.

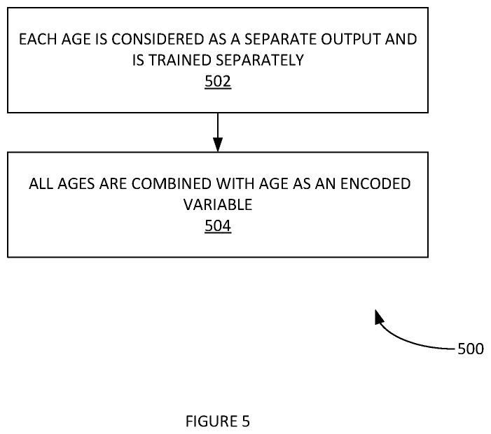

FIG. 5 illustrates an example process 500 for feeding the data into an ML model, according to some embodiments. Each step can be utilized separately for feeding the data into the model in some examples. In step 502, each age is considered as a separate output and is trained separately. In step 504, all ages are combined with age as an encoded variable.

Several methods of discrete variable encoding, including one-hot-encoding, thermometer encoding (e.g. thermometer encoding is like one-hot encoding, but it represents magnitude instead of a categorical variable), and binary encoding are used and the most suitable for each dataset is selected. Encoding variables in a machine learning framework can be used to change the format of a variable in a way that it improves the accuracy when feeding into the machine learning model.

Discrete variable encoding methods, including one-hot-encoding, thermometer encoding, and binary encoding can be tested. One-hot encoding can generate a column for each of the values of the discrete variable, with the true column of each specific row being one and the rest of the columns being zero.

Thermometer encoding can create the columns similar to one-hot encoding, however, instead of just making the related column value equal to one, it makes all the earlier ones equal to one, too. For example, if the first category has all columns zero, the second category has the first column equal to one, and the third category has the first and second columns equal to one.

In binary encoding the number of columns are only as many as the digits of the binary number equivalent to the number of categories. Then, each category 1 to n is encoded as a binary number, with the digits replaced in the new columns.

Using these methods, the most suitable method is selected for each dataset by cross-validation. As our general dataset expands further hyperparameter tuning can be conducted regularly. Therefore, although a certain encoding method may be the most suitable for a period of time, there is a chance that another variable encoding method may prove more effective in the future as the data expands.

In some embodiments, a combination of three artificial neural network models can be used as one of the elements of a final combined model. As each artificial neural network model includes many hyperparameters, a hyperparameter tuning procedure can be utilized as well.

An Adam Optimizer can be used in specified ML models as well. The learning rate, initial decay rates, and epsilon can be tuned. The ML model can be a combination of three artificial neural networks with different numbers of layers. A dropout method can be used for regularization. The numbers of layers, numbers of units in each layer, activation functions, number of mini-batches and epochs, and the dropout regularization rates are tuned as well. 5-fold cross-validation is used for hyperparameter tuning and test set for monitoring the model errors. Error can be reflected as a mean squared error (MSE) and/or Huber loss. Huber loss is a combination of squared and absolute loss terms that is less sensitive to outliers in comparison to MSE. This can be useful for compressive strength prediction, so that a few very low compressive strength outputs do not cause misinterpretation of a model that is in fact performing well. To improve performance of the ML model as well as computational efficiency, supervised feature selection is performed. This can include the process of selecting a subset of relevant features (e.g. variables, predictors) for use in ML model construction

Forward selection, and perturbation rank can be utilized in some ML models. Feature selection can be implemented through a 5-fold cross-validation, at the same time as hyperparameter tuning. Forward selection can involve starting with no variables in the model, testing the addition of each variable using a chosen model fit criterion, adding the variable (if any) whose inclusion gives the most statistically significant improvement of the fit, and repeating this process until none improves the model to a statistically significant extent.

To remedy the excessive computational burden of this process, subsets of features were tested instead of individual features. Other than the artificial neural networks models, random forests, gradient boosting, and non-linear regression models can be applied to the data (e.g. see supra for data sources/types, etc.). As the result of random forest and gradient boosting can be used in some examples and their results can be combined with the artificial neural networks predictions as an average which improves the prediction error.

In step 604, process 600 can implement a classification predication model. To improve the prediction accuracy, a number of classification models are created and utilized. Classification models can perform an additional round of testing on the selected mixes evaluating if they are recognized among the range of a desired output. For example, the concrete mixes can be categorized into two classes of over 24 in. slump and under. Many classification models can be utilized, including, inter alia: artificial neural networks, random forest methods, gradient boosting methods, logistic regression methods, support vector machines (SVM) methods, Gaussian naïve Bayes methods, discriminant analysis methods, and K-nearest-neighbor (KNN) methods. These methods can be used to develop classification models.

In one example, a naïve Bayes by cross-validation can be used as a classification method. In one example, a combination of KNN and Quadratic Discriminant Analysis (QDA) methods can be utilized for classification.

As an additional round of examination with classification models can be used to improve the accuracy and precision of the prediction. Using different models can also be used to improve the result knowing models learn in different ways and therefore they have a level of independence between their results. This can be used to lower the probability of a wrong prediction when several models have confirmed a certain untested mix. It is noteworthy that for different levels of classifying outputs a different model might be useful.

In order to evaluate the results of the model, the appropriate metric or metrics should be selected from among a variety of statistical measures. Example useful methods and metrics include, inter alia: accuracy, precision, recall, F1-score, confusion matrix, and ROC curve.

Precision can be determined and analyzed as a metric. The sparsity of the class of interest in the data can be a reason to evaluate precision. In other words, the data may be heavily imbalanced. For example, 7-day compressive strength>4,500 psi may be true about only 10% of the data or less. Therefore, process 600 can utilized the precision metric (e.g. what is the probability of correctness when a mix is classified as positive, rather than making sure that we find all the members of the positive class, etc.).

Another approach used to deal with the imbalance of the data is synthetic minority oversampling technique (SMOTE). This is an oversampling technique which avoids overfitting unlike many others.

In step 606, process 600 can implement Bayesian optimization. Bayesian optimization is an optimization method used when the objective function is a black box function. Getting samples from this function may be expensive and therefore the queries can be made with caution. So, the question is how to query this function to get to the optimal point within few queries. In a nutshell, at each step, the model selects the next point based on the optimum of the acquisition function. Acquisition function contains the information from mean as well as variance of the space. Hence, it can show us where we might make an improvement based on the current knowledge of the objective function. This process continues until the selected points converge. The Bayesian optimization model suits the problem of making concrete mixes due to the expensive procedure of making mixes and running concrete tests on samples.

The Bayesian optimization model provides a framework to manage exploration versus exploitation. Bayesian optimization model can be used to take advantage of the knowledge of the best points while exploring an unseen space. Focusing on exploration, a goal can be to lower the variance, therefore, the areas with high variance are explored. To focus on exploitation, process 600 can slightly move from the best point it has seen in search of better points. The acquisition function is the means to balance these two goals.

Bayesian optimization can use Gaussian process as the estimate of the prior distribution. It is noted that a Gaussian process does not render a remarkable performance in comparison to more complicated regression methods such as artificial neural networks and random forests for modeling the data. Therefore, in these cases, process 600 can use a regression prediction model (e.g. see supra). To be applicable to Bayesian optimization, a regression model can both make predictions and provide a measure of uncertainty over those predictions. In one example regression model, when using a combination of several models, the standard deviation of predictions of different models is a good measure of uncertainty.

Process 600 can use a variety of acquisition functions to use. This can include, inter alia: Probability of Improvement (PI), Expected Improvement (EI), Upper Confidence Bound (UCB), and Thompson sampling. While all of these methods can converge given the right model being used, in one example embodiments, EI can be applied in our work so far.

In some examples, Monte Carlo optimization and cross entropy optimization methods can be applied. Cross entropy can be used to find the optimum point with fewer samples than the Monte Carlo optimization. Monte Carlo optimization is the process of randomly generating input data from a probability distribution function and finding the optimum point based on the set of random inputs.

Cross entropy can be a sophisticated Monte Carlo method that is used as a sampling method used for rare event simulation. In one example, the optimization problems can be reformulated as a rare event optimization and cross entropy can be applied to the present optimizations. In the process of cross entropy optimization, the sampling distribution can be updated regularly (e.g. at a specified intervals during the application of process 600, etc.) to reach a sampling distribution where the probability of the rare event occurring is high.

The objective function may depend heavily on the expectation from the model. For example, if the objective is to maximize early age strength while maintaining a minimum slump of 26 in. and a minimum 7-day compressive strength of 5,000 psi, the following objective function (or similar) can be defined as follows:

Max f1+f2−relu(5,000−f7)*M7−relu(26−slump)*Ms,

where f1, f2, and f7 represent 1-day, 2-day, and 7-day compressive strength, respectively. M7 and Ms are the costs of f7 and slump being lower than the desired amount. relu(x) is a function that is equal to zero when x<0 and equal to x everywhere else.

### Example Computer Architecture and Systems

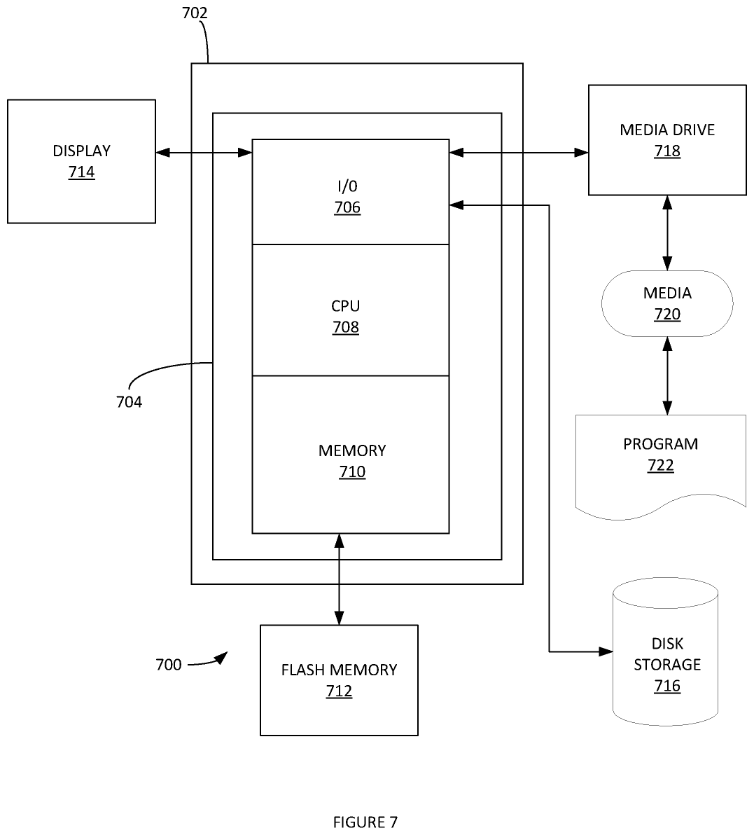

FIG. 7 depicts an exemplary computing system 700 that can be configured to perform any one of the processes provided herein. In this context, computing system 700 may include, for example, a processor, memory, storage, and I/O devices (e.g., monitor, keyboard, disk drive, Internet connection, etc.). However, computing system 700 may include circuitry or other specialized hardware for carrying out some or all aspects of the processes. In some operational settings, computing system 700 may be configured as a system that includes one or more units, each of which is configured to carry out some aspects of the processes either in software, hardware, or some combination thereof.

FIG. 7 depicts computing system 700 with a number of components that may be used to perform any of the processes described herein. The main system 702 includes a motherboard 704 having an I/O section 706, one or more central processing units (CPU) 708, and a memory section 710, which may have a flash memory card 712 related to it. The I/O section 706 can be connected to a display 714, a keyboard and/or other user input (not shown), a disk storage unit 716, and a media drive unit 718. The media drive unit 718 can read/write a computer-readable medium 720, which can contain programs 722 and/or data. Computing system 700 can include a web browser. Moreover, it is noted that computing system 700 can be configured to include additional systems in order to fulfill various functionalities. Computing system 700 can communicate with other computing devices based on various computer communication protocols such a Wi-Fi, Bluetooth® (and/or other standards for exchanging data over short distances includes those using short-wavelength radio transmissions), USB, Ethernet, cellular, an ultrasonic local area communication protocol, etc.

Electrical resistivity examples are now discussed. It is noted that a device is used that utilizes AC impedance technique to measure the electrical resistivity of concrete cylinders. Electrical resistivity could then be correlated with important durability parameters such as permeability and diffusivity as well as pore network characteristics such as pore size and their connectivity.

Compression test examples are now discussed. It is noted that concrete cylinders or cubes are tested under compression using hydraulic systems at a specific load or displacement rate to measure the ultimate load capacity of concrete needed to experimentally crush concrete samples. This ultimate load divided by the surface area of the sample is reported as the concrete strength and is usually measured and recorded at different ages e.g. 1-day, 7-day, 28-day, etc.

Some examples of fresh properties of concrete are slump, temperature, unit weight, setting time, air content, etc.

Slump examples are now discussed. It is noted that slump test measures the flowability of concrete. For this test, a standard cone is used and filled with freshly mixes concrete in 3 layers. After consolidating each layer by tapping with a standard rod 25 times, the top surface is made flush with the top of the cone and then the cone is lifted vertically. The slump of the concrete is measured by measuring the distance from the top of the slumped concrete to the level of the top of the slump cone. In case of self-consolidating concrete, the diameter of the slumped concrete is measured in various directions and the average value is recorded as the slump.

Temperature of fresh concrete can be measured after mixing using a special concrete thermometer.

Unit weight examples are now discussed. It is noted that freshly mixed concrete is poured into a mold of specific volume in three layers. After consolidating each layer by tapping with a standard rod 25 times, the top surface is leveled, and the net weight of concrete is measured. Unit weight of concrete is then calculated by dividing the weight by the known volume of the mold.

Setting time examples are now discussed. It is noted that setting time of concrete is measured by using a device that penetrates a needle of various certain sizes into fresh concrete and measures the applied load. Such measurements are taken at various time intervals after mixing for several hours, until the stress calculated by dividing the load by the cross-sectional area of the needle surpasses a certain level.

Air content examples are now discussed. It is noted that fresh concrete contains a certain level of air reported as a volume percentage. A specific standard device is used to measure the air content by pumping air into a chamber and then releasing it into fresh concrete contained in an enclosed container. The air content is then read from the gauge.

### CONCLUSION

Although the present embodiments have been described with reference to specific example embodiments, various modifications and changes can be made to these embodiments without departing from the broader spirit and scope of the various embodiments. For example, the various devices, modules, etc. described herein can be enabled and operated using hardware circuitry, firmware, software or any combination of hardware, firmware, and software (e.g., embodied in a machine-readable medium).

In addition, it will be appreciated that the various operations, processes, and methods disclosed herein can be embodied in a machine-readable medium and/or a machine accessible medium compatible with a data processing system (e.g., a computer system), and can be performed in any order (e.g., including using means for achieving the various operations). Accordingly, the specification and drawings are to be regarded in an illustrative rather than a restrictive sense. In some embodiments, the machine-readable medium can be a non-transitory form of machine-readable medium.

What is claimed by United States patent:

1. A method of optimizing a concrete formulation comprising:
providing a historical data set of previously sampled concrete mixes;
with the historical data set, generating and validating a set of machine learning models;
obtaining data from a concrete mix for input into a computer system that generates a machine learning model, wherein the data is obtained by:
obtaining three samples to be tested at each of a 1-day age of the concrete mix, a 2-day of the concrete mix, a 7-day of the concrete mix, a 14-day of the concrete mix, a 28-day of the concrete mix and a 56-day of the concrete mix;
implementing a resistivity test and a compression test on each concrete sample, wherein for each sample resistivity test and compression test:
digital recording the resistivity test result and compression test result in a computer readable medium;
implementing a fresh properties test on each sample and digitally recording the results of the fresh properties test in the computer readable medium;
generating a test result dataset comprising each result of each digital recording of each resistivity test result, each compression test result, and each fresh properties test result;
with the test result dataset:
applying the set of machine learning models the test result dataset and generating a prediction output for at least one concrete mixing parameter of a new concrete mix.

2. The method of claim 1, wherein the fresh properties test comprises implementing a slump test on each sample.

3. The method of claim 2, wherein the fresh properties test comprises implementing a density test on each sample.

4. The method of claim 3, wherein the fresh properties test comprises implementing an air content test on each sample.

5. The method of claim 4, wherein the fresh properties test comprises implementing a moisture content test on each sample.

6. The method of claim 5, wherein the fresh properties test comprises implementing a temperature content test on each sample.

7. The method of claim 6, wherein the fresh properties test comprises implementing a humidity during mixing test on each sample.

8. The method of claim 1, wherein the set of machine learning models comprises a plurality of regression prediction models.

9. The method of claim 1, wherein the set of machine learning models comprises a plurality of classification prediction models that is used to calculate the at least one concrete mixing parameter of the new concrete mix.

10. The method of claim 1, wherein the set of machine learning models comprises a plurality of Bayesian optimization prediction models that is used to calculate the at least one concrete mixing parameter of the new concrete mix.

11. The method of claim 1, wherein the set of machine learning models comprises an ensemble machine learning prediction model that is used to calculate the at least one concrete mixing parameter of the new concrete mix.

12. The method of claim 11 further comprising:
using a weighted average of each individual prediction of each machine learning model in the ensemble machine learning model as a final prediction for the at least one concrete mixing parameter of the new concrete mix.

13. The method of claim 12 further comprising:
calculating a set of test set errors to generate a power value of the ensemble model.

14. The method of claim 13, wherein a cross-validation process is used to estimate at least one hyperparameter and to select one or more relevant features.

15. The method of claim 14, wherein the ensemble machine learning prediction model comprises a regression prediction model, a Bayesian prediction model, and a classification prediction model.

16. A method of optimizing a concrete formulation comprising:
pulling a set of customer data and augment it with data sets designed to optimized machine learning operations;
selecting specified machine learning techniques from a specified machine learning database;
taking into account a set of worksite context parameters; and
optimizing and adjusting a concrete mix in real time to be able to deliver concrete products that meets requirements of project.
